(19) United States
(12) Patent Application Publication
(10) Pub. No.: US 2016/0074210 A1
(43) Pub. Date: Mar. 17, 2016

(54) COLD/HOT COMPRESSOR THERAPY DEVICE

(71) Applicant: Ma-Li CHEN, New Taipei City (TW)

(72) Inventor: Ma-Li CHEN, New Taipei City (TW)

(21) Appl. No.: 14/718,208

(22) Filed: May 21, 2015

(30) Foreign Application Priority Data
Sep. 15, 2014 (TW) 103216339

(51) Int. Cl. A61F 7/00

(52) U.S. Cl. CPC A61F 7/007

(57) ABSTRACT

A cold/hot compressor therapy device includes a contact module, a heating/cooling module and a housing module. The contact module includes a contact wall to be in contact with a user, and a surrounding wall extending from the contact wall, cooperating with the contact wall to define an accommodating space, and having an annular flange. The heating/cooling module is disposed in the accommodating space to contact the contact wall and is operable to absorb or to provide heat. The housing module includes a bottom wall and a limiting wall. The contact module projects outwardly of the housing module through the bottom wall. The annular flange is movable toward and away from the contact wall so that the contact module is movable toward and away from the bottom wall.

### CROSS-REFERENCE TO RELATED APPLICATION

This application claims priority of Taiwanese Utility Model Application No. 103216339, filed on Sep. 15, 2014.

### FIELD

The disclosure relates to a therapy device, and more particularly to a cold/hot compressor therapy device.

### BACKGROUND

Hot compressor therapy and cold compressor therapy are the most common forms of treatment for alleviating inflammation and/or swelling caused by injuries (such as sprain), allergies and/or anemia. In addition, symptoms caused by the same, such as skin rash or pain, can be prevented using the hot/cold compressor therapy. Some conventional heating/cooling devices may contain water or chemical solution and need to he stored in a heater/refrigerator for a period of time in advance to obtain heating/cooling ability, so that when urgent use of the same is called for, the wait time of a user may be significantly prolonged, often even having the device removed prematurely from storage so that less-than-optimal therapeutic results are undesirably produced. Moreover, during application, the conventional heating/cooling device may need to be pressed forcefully against the injury, which may result in discomfort of the patient.

### SUMMARY

Therefore, an object of the disclosure is to provide a cold/hot compressor therapy device that can alleviate at least one of the drawbacks of the prior art.

According to one aspect of the disclosure, a cold/hot compressor therapy device includes a contact module, a heating/cooling module, a housing module and a resilient module. The contact module includes a contact wall that is adapted to be in contact with a user, and a surrounding wall that extends transversely from a periphery of the contact wall, that cooperates with the contact wall to define an accommodating space, and that has an annular flange which extends from a periphery of the surrounding wall outwardly of the accommodating space and oppositely of the contact wall. The heating/cooling module is disposed in the accommodating space to contact an inner surface of the contact wall and is operable to absorb heat from or to provide heat to the contact wall. The housing module surrounds the contact module and includes a bottom wall, and a limiting wall spaced apart from the bottom wall. The contact wall and the surrounding wall project outwardly of the housing module through the bottom wall. The annular flange of the contact module is disposed between an inner surface of the bottom wall and the limiting wall, and is movable toward and away from the inner surface of the contact wall so that the contact wall and the surrounding wall are movable toward and away from an outer surface of the bottom wall. There resilient module is disposed between the bottom wall and the limiting wall to bias the annular flange to move toward the inner surface of the bottom wall.

According to another aspect of the disclosure, a cold/hot compressor therapy device includes a housing module, a contact module, a heating/cooling module and a resilient module. The housing has a bottom cover. The contact module is disposed in the housing module and includes a contact wall that is adapted to contact a user, and a surrounding wall that extends transversely from a periphery of the contact wall and that cooperates with the contact wall to define an accommodating space. The contact wall and the surrounding wall project outwardly from the housing module through the bottom cover, and the contact wall is movable toward and away from an outer surface of the bottom cover. The heating/cooling module is disposed in the accommodating space to contact an inner surface of the bottom wall and is operable to absorb heat from or to provide heat to the contact wall. The resilient module is disposed in the housing module to bias the contact module to move toward an inner surface of the bottom cover so that the contact wall is resiliently movable toward and away from the outer surface of the bottom cover.

### BRIEF DESCRIPTION OF THE DRAWINGS

Other features and advantages of the disclosure will become apparent in the following detailed description of the embodiment with reference to the accompanying drawings, of which:

FIG. 1 is an exploded perspective view illustrating an exemplary embodiment of a cold/hot compressor therapy device according to the disclosure; and

FIG. 2 is a sectional view of the exemplary embodiment.

### DETAILED DESCRIPTION

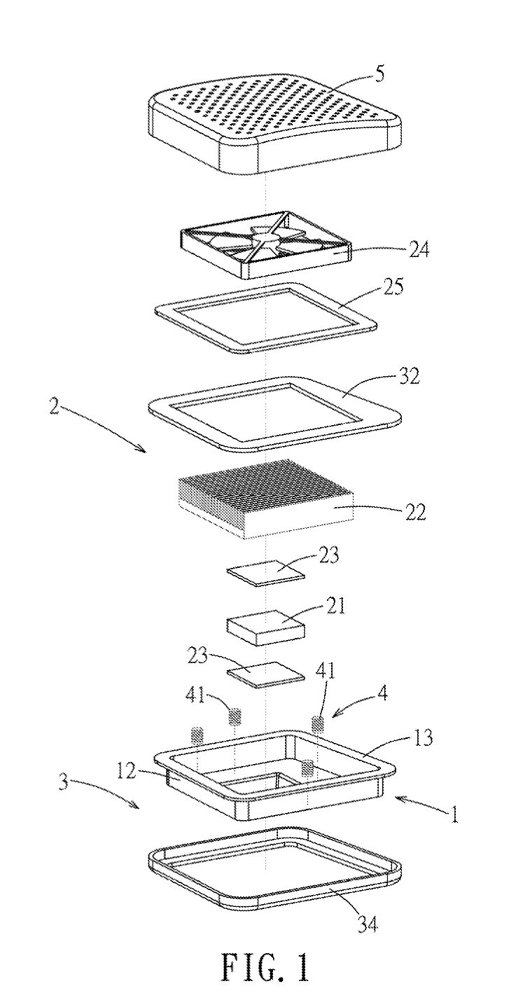

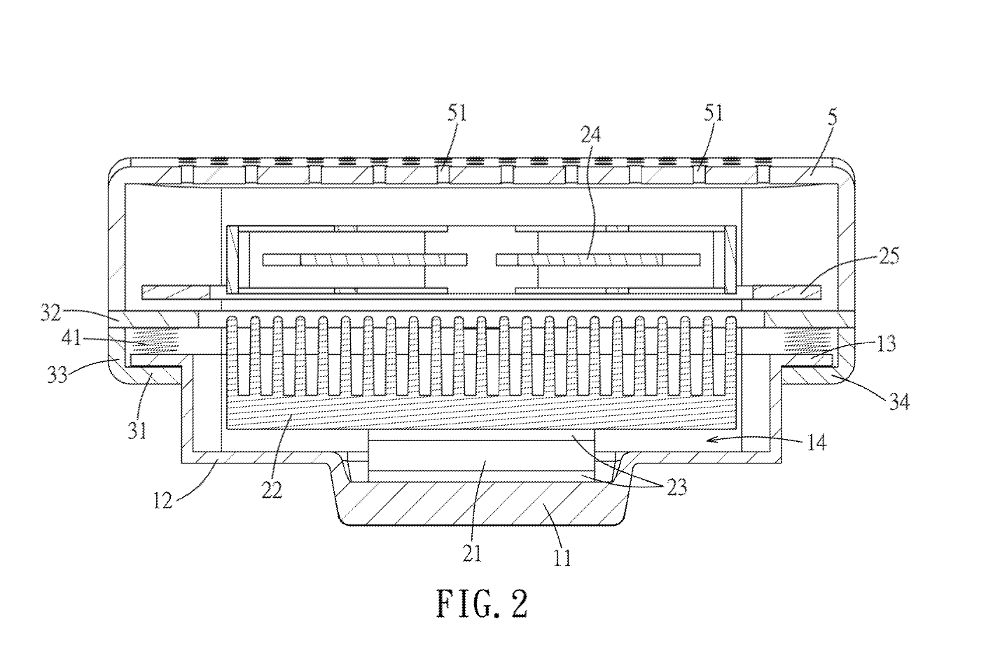

Referring to FIGS. 1 and 2, the exemplary embodiment of a cold/hot compressor therapy device according to the present disclosure is shown to include a contact module 1, a heating/cooling module 2, a housing module 3 and a resilient module 4.

The contact module 1 includes a contact wall 11 that is adapted to be in contact with a user (or a patient), and a surrounding wall 12 that extends transversely from a periphery of the contact wall 11, that cooperates with the contact wall 11 to define an accommodating space 14, and that has an annular flange 13 extending from a periphery of the surrounding wall 11 outwardly of the accommodating space 14 and oppositely of the contact wall 11. The contact module 1 may be made of a thermally-conductive material. In this embodiment, the contact module 1 may be made of aluminum, but other materials having superior thermal conductivity, such as copper and ferrite, may be used in other embodiments according to the present disclosure.

The heating/cooling module 2 is disposed in the accommodating space 14 to contact an inner surface of the contact wall 11, and is operable to absorb heat from or to provide heat to the contact wall 11. In this embodiment, the heating/cooling module 2 includes a thermoelectric chip 21 attached to the inner surface of the contact wall 11, and a heat sink 22 attached to the thermoelectric chip oppositely of the contact wall 11. The heating/cooling chip 21 may be switchable between a cooling mode, where the thermoelectric chip 21 is operable to absorb heat from the contact wall 11 for cooling the user's skin in contact with the contact wall 11, and a heating mode, where the thermoelectric chip 21 is operable to provide heat to the contact wall 11 for heating the user's skin in contact with the contact wall 11. The heating/cooling module 2 further includes an annular control circuit board 25 that is electrically coupled to the thermoelectric chip 21 and that is operable to switch the thermoelectric chip 21 between the cooling mode and the heating mode. Here, the heating/cooling module 2 as illustrated in FIG. 1 may, for instance, further include a pair of heat-conductive adhesive layers 23, one for attaching the thermoelectric chip 21 onto the contact wall 11 and the other for attaching the heat sink 22 onto the thermoelectric chip 21. In certain embodiments, the heating/cooling module 2 may further include a fan 24 disposed on top of the heat sink 22 for dissipating heat.

The housing module 3 surrounds the contact module 1 and includes a bottom wall 31 and a limiting wall 32 spaced apart from the bottom wall 31. As shown in FIG. 2, the contact wall 11 and the surrounding wall 12 of the contact module 1 project outwardly of the housing module 3 through the bottom wall 31 of the housing module 3. The annular flange 13 of the contact module 1 is disposed between an inner surface of the bottom wall 31 and the limiting wall 32, and is movable toward and away from the inner surface of the bottom wall 31, so that the contact wall 11 and the surrounding wall 12 are movable toward and away from the an outer surface of the bottom wall 31. In this embodiment, the housing module 3 is configured in a substantially rectangular shape and further includes a bottom cover 34 having the bottom wall 31, and an annular side wall 33 extending transversely and perpendicularly from a periphery of the bottom wall 31 to the limiting wall 32 and connected to the limiting wall 32 (e.g., using adhesives such as silicone). The bottom cover 34 of the housing module 3 surrounds the contact module 1. As shown in FIG. 2, in this embodiment, the bottom wall 31 and the limiting wall 32 may be configured as parallel annular plates. It should be noted that the housing module 3 may further include a top cover 5 that is disposed above and connected to the limiting wall 32 and that surrounds the heating/cooling module 2. In this embodiment, the top cover 5 covers the fan 24 and the control circuit board 25. The top cover 5 may be formed with a plurality of vent holes 51.

The resilient module 4 is disposed in the housing module 3 to bias the contact module 1 to move toward an inner surface of the bottom cover 34 so that the contact wall 11 is resiliently movable toward and away from an outer surface of the bottom cover 34. To be specific, in this embodiment, the resilient module 4 is disposed between the bottom wall 31 and the limiting wall 32 for biasing the annular flange 13 to move toward the inner surface of the bottom wall 31. In this embodiment, the resilient module 4 includes a plurality of spaced-apart resilient members 41 (four, for instance, as illustrated in FIG. 1) disposed between the annular flange 12 and the limiting wall 31. The resilient members 41 may be, but are not limited to, compression springs.

When in use, the contact wall 11 of the contact module 1 may be in direct contact with the user's skin, and the thermoelectric chip 21 may be operable to absorb heat from or to provide heat to the contact wall 11 without the need for waiting. In addition, when the cold/hot compressor therapy device is pressed against the user's skin, the resilient module 4 may convert at least partially a pressure applied onto the cold/hot compressor therapy device into potential energy thereof by virtue of the elasticity of the resilient members 41, so that any pain caused by such pressure may be alleviated. When the cold/hot compressor therapy device is removed from the user's skin, the biasing force provided by the resilient module 4 causes the annular flange 13 to move toward the inner surface of the bottom wall 31, and the contact wall 11 and the surrounding wall 12 to move away from the outer surface of the bottom wall 31.

While the disclosure has been described in connection with what is considered the exemplary embodiment, it is understood that this disclosure is not limited to the disclosed embodiment but is intended to cover various arrangements included within the spirit and scope of the broadest interpretation so as to encompass all such modifications and equivalent arrangements.

What is claimed is:

1. A cold/hot compressor therapy device, comprising:
a contact module including a contact wall that is adapted to be in contact with a user, and a surrounding wall that extends transversely from a periphery of said contact wall, that cooperates with said contact wall to define an accommodating space, and that has an annular flange which extends from a periphery of said surrounding wall outwardly of said accommodating space and oppositely of said contact wall;
a heating/cooling module that is disposed in said accommodating space to contact an inner surface of said contact wall and that is operable to absorb heat from or to provide heat to said contact wall;
a housing module surrounding said contact module and including a bottom wall, and a limiting wall spaced apart from said bottom wall, said contact wall and said surrounding wall projecting outwardly of said housing module through said bottom wall, said annular flange of said contact module being disposed between an inner surface of said bottom wall and said limiting wall, and being movable toward and away from said inner surface of said bottom wall so that said contact wall and said surrounding wall are movable toward and away from an outer surface of said bottom wall; and
a resilient module that is disposed between said bottom wall and said limiting wall to bias said annular flange to move toward said inner surface of said bottom wall.

2. The cold/hot compressor therapy device set forth in claim 1, wherein said contact module is made of a thermally-conductive material.

3. The cold/hot compressor therapy device set forth in claim 2, wherein said heating/cooling module includes a thermoelectric chip attached to said inner surface of said contact wall, and a heat sink attached to said thermoelectric chip oppositely of said contact wall.

4. The cold/hot compressor therapy device set forth in claim 3, wherein:
said housing module further includes a bottom cover having said bottom wall, and an annular side wall extending transversely from a periphery of said bottom wall to said limiting wall and connected to said limiting wall; and
said bottom wall and said limiting wall are configured as parallel annular plates.

5. The cold/hot compressor therapy device set forth in claim 3, wherein said heating/cooling module further includes a fan disposed on said heat sink for dissipating heat.

6. The cold/hot compressor therapy device set forth in claim 5, wherein:
said thermoelectric chip is switchable between a cooling mode, where said thermoelectric chip is operable to absorb heat from said contact wall, and a heating mode, where said thermoelectric chip is operable to provide heat to said contact wall; and
said heating/cooling module further includes a control circuit board that is electrically coupled to said thermoelectric chip and that is operable to switch said thermoelectric chip between the cooling mode and the heating mode.

7. The cold/hot compressor therapy device set forth in claim 6, wherein said resilient module includes a plurality of spaced-apart resilient members disposed between said annular flange and said limiting wall.

8. The cold/hot compressor therapy device set forth in claim 7, wherein said housing module further includes a top cover that is disposed above and connected to said limiting wall and that covers said fan and said control circuit board, said top cover being formed with a plurality of vent holes.

9. A cold/hot compressor therapy device, comprising:
a housing module having a bottom cover;
a contact module disposed in said housing module and including a contact wall that is adapted to contact a user, and a surrounding wall that extends transversely from a periphery of said contact wall and that cooperates with said contact wall to define an accommodating space, said contact wall and said surrounding wall projecting outwardly from said housing module through said bottom cover and said contact wall being movable toward and away from an outer surface of said bottom cover;
a heating/cooling module that is disposed in said accommodating space to contact an inner surface of said contact wall and that is operable to absorb heat from or to provide heat to said contact wall; and
a resilient module disposed in said housing module to bias said contact module to move toward an inner surface of said bottom cover so that said contact wall is resiliently movable toward and away from said outer surface of said bottom cover.

10. The cold/hot compressor therapy device set forth in claim 9, wherein said bottom cover includes a bottom wall, and an annular side wall extending transversely from a periphery of said bottom wall, said contact wall and said surrounding wall extending through said bottom wall, said housing module further having a limiting wall disposed above said bottom cover and connected to said annular side wall oppositely of said bottom wall, and a top cover disposed above and connected to said limiting wall, said surrounding wall of said contact module having an annular flange which extends from a periphery of said surrounding wall outwardly of said accommodating space and oppositely of said contact wall, said annular flange being disposed between an inner surface of said bottom wall and said limiting wall and being movable toward or away from said inner surface of said bottom wall.

11. The cold/hot compressor therapy device set forth in claim 10, wherein said resilient module is disposed between said limiting wall and said annular flange.

12. The heat/cooling therapy device set forth in claim 11, wherein said bottom cover surrounds said contact module, and said top cover surrounds said heating/cooling module.
